(19) United States
(12) Patent Application Publication
(10) Pub. No.: US 2022/0369977 A1
(43) Pub. Date: Nov. 24, 2022

(54) INTELLIGENT PSYCHOLOGICAL ASSESSMENT AND INTERVENTION SYSTEM AND METHOD BASED ON AN INDEPENDENT SPACE

(71) Applicant: SUZHOU HB AI TECHNOLOGY RESEARCH&DEVELOPMENT CO., LTD., Suzhou, Jiangsu (CN)

(72) Inventors: Erdong WANG, Suzhou, Jiangsu (CN); Jinwen SHEN, Suzhou, Jiangsu (CN); Donghai XUE, Suzhou, Jiangsu (CN); Zhifeng ZHAO, Suzhou, Jiangsu (CN); Qing JI, Suzhou, Jiangsu (CN)

(21) Appl. No.: 17/771,809

(22) PCT Filed: Sep. 28, 2020
(86) PCT No.: PCT/CN2020/118301

§ 371 Date: Apr. 25, 2022

(30) Foreign Application Priority Data
Oct. 25, 2019 (CN) 201911022217.1

(51) Int. Cl. A61B 5/16; G16H 10/20; G16H 70/20; G16H 50/70; G16H 20/70

(52) U.S. Cl. CPC A61B 5/165; G16H 10/20; G16H 70/20; G16H 50/70; G16H 20/70; A61B 2560/0247

(57) ABSTRACT

An intelligent psychological assessment and intervention system based on an independent space, comprising a psychological intervention cabin and a central database which achieves data interaction with the psychological intervention cabin. The psychological intervention cabin comprises a housing, a data acquisition module, a data processing module, and an intervention module; the data acquisition module acquires physiological and psychological data of a subject; the data processing module performs operations on the acquired data, establishes a multi-dimensional state point of the subject according to a mathematical model of the data processing module, matches it with data in the central database, finds a suitable intervention procedure for the subject, and guides the intervention module to carry out intervention; in the intervention process, the data acquisition module continuously acquires the physiological and psychological data of the subject, and the data processing module forms a new multi-dimensional state point according to new data and re-matches it to adjust the intervention procedure. By repeating this cycle, the intervention procedure is continuously adjusted in the intervention process to seek a most suitable intervention solution for the subject, and an optimal intervention effect is achieved.

### FIELD OF THE INVENTION

The invention relates to artificial intelligence, big data, psychological services and other related fields, and specifically relates to an intelligent psychological assessment and intervention system and a method thereof.

### BACKGROUND

With the rapid development of society, the pressure of work and life is increasing. Various psychological problems are emerging, which have a huge impact on daily life. People start to pay attention to mental health issues while paying attention to physical health.

At present, relative to the huge demand for social mental health, professional psychological assessment and intervention resources are seriously insufficient, mainly concentrated on the insufficient staffing of psychological counseling centers or psychological counselors. Because people are generally subjective, prejudiced, and narrow, the acquisition of background information and subsequent judgements are inevitably subjective and one-sided. As professional psychological assessors, psychological counselors or interventionists need to abandon personal subjectivity and one-sidedness and evaluate people's psychological conditions from an objective perspective. It takes years of training and experience accumulation before taking up the job to have a high level of professionalism, which results in the slow output and shortage of psychological counseling or intervention talents.

In addition, when facing mental health issues, due to discrimination and colored vision in the real society, the subjects often resist going to special psychological counseling or intervention sites, such as hospitals or psychological counseling centers to seek professional services. At the same time, due to a lack of mental health knowledge, a correct understanding of psychological problems has not been formed. When facing psychological pressure or emotional crisis, people are often passive and helpless, waiting for psychological problems to aggravate or get even worsen, resulting in increased family and social burdens. Therefore, in daily life, the establishment of a psychological assessment and intervention system that meets privacy protection needs and can cover large-scale populations has become an important way to solve privacy issues in psychological counseling and intervention services.

In addition to the issues mentioned above, in the traditional psychological assessment and intervention process, the psychological counseling or intervention process follows a fixed or certain process to inquire and intervene with the subjects. However, various real subjects' problems are different from each other. If the psychological assessment and intervention of different subjects are carried out according to a fixed process, the intervention process may be unscientific, and the final result may be not ideal. Therefore, it is an important solution to find a process that can be changed according to the specific conditions of different subjects to conduct targeted psychological counseling, assessment, and intervention to improve the success rate of psychological assessment and intervention.

### SUMMARY

The present invention provides an independent space-based intelligent psychological assessment and intervention system and method. The intelligent psychological assessment and intervention system learns a large number of counseling cases and has the functions of data acquisition and data analysis. The system can replace the role of psychological counseling, assessment, or intervention staff, relieving the pressure of the shortage of psychological counselors. The system is based on an independent and relatively private space to ensure the privacy of the subjects. In addition, more importantly, the system integrates big data analysis and matching, uses various sensors to acquire data from users, analyzes and models the acquired data, and continuously adjusts the intervention process according to the characteristics of different subjects. As such, the intervention process is more targeted, and the intervention effect is more ideal. It should be noted that the terms used in the technology of this patent application to describe facilitating in changing of the psychological state or understanding of the psychological state, such as counseling, assessment, intervention, etc., unless otherwise specified, have the same or similar meanings and can be substituted for each other.

To solve the above-mentioned existing technical problems, the present invention provides an intelligent psychological assessment and intervention system based on an independent space. The system includes a psychological intervention cabin and a central database capable of achieving data interaction with it. The data processing module is connected to the central database, the data acquisition module and the intervention module. The psychological intervention cabin includes a housing, a data acquisition module disposed in the housing, an internal or external data processing module, and an intervention module. The central database stores a number of sample individuals and the corresponding intervention plans. The data acquisition module acquires the relevant physiological and psychological data of the subject and transmits the data to the data processing module. The data processing module fuses the data transmitted by the acquisition module according to its mathematical model, matches it with the samples in the central database, calls the intervention plan corresponding to the matched sample, and guide the intervention module to intervene with the subject according to the intervention plan.

Wherein, the mathematical model of the data processing module is a multi-dimensional spatial coordinate system. The data processing module establishes the subject's multi-dimensional state point in the multi-dimensional spatial coordinate system according to the data acquires by the data acquisition module and matches it to the multi-dimensional state points in the central database to find the same or similar matching point.

In the intervention procedure, the data acquisition module continuously acquires information from the subject and transmits the acquired information to the data processing module. The data processing module forms a new multi-dimensional state point about the subject based on the received data, matches the corresponding new intervention procedure in the central database, and guides the intervention module to adjust the intervention procedure, iteratively, thereby continuously adjusting the intervention procedure during the intervention process and find the most suitable intervention plan for the subject.

The psychological intervention cabin is an independent space that is independently set up and has a confined space.

Further, the data processing module is connected to the central database, the data acquisition module, and the intervention module by wireless and/or wired communications. The central database stores a plurality of multi-dimensional state points of different individuals, and intervention plans for different individuals. The individual's multi-dimensional state point refers to selecting different indicator parameters to establish a multi-dimensional coordinate system, depicting the individual's multi-dimensional state point in the multi-dimensional coordinate system according to the individual's multiple indicator values, and forming the individual's data model.

The data acquisition module is responsible for acquiring the subject's relevant information and/or ambient information in the housing of the psychological intervention cabin.

The data processing module at least includes an arithmetic unit. The arithmetic unit fuses the information acquired by the data acquisition module according to its mathematical model, establishes the subject's multi-dimensional state point, and performs operations, thereby calling a suitable intervention plan pre-stored in the central database according to the operation result. The data processing module may also include a memory unit. The memory unit receives the subject's relevant information and/or ambient information in the housing acquired by the data acquisition module, and then the arithmetic unit reads data from the memory unit for processing.

The operation process of the data processing module refers to that the arithmetic unit matches the subject's multi-dimensional state point to a plurality of multi-dimensional state points pre-stored in the central database, finds the same or similar point to the subject's multi-dimensional state point, and determines whether the subject's multi-dimensional state point is in a depressed state, a normal state, or an excited state. If it is in a depressed state, the arithmetic unit guides the intervention module to perform intervention on the subject according to the intervention procedure at this point. If it is in a normal state, the subject is empowered. If it is in an excited state, the subject is prompted to leave the cabin immediately. The depressed state, normal state and excited state mentioned herein refer to: determining whether the subject is in a negative state of mind, a peaceful state of mind, or a positive state of mind based on the multi-dimensional data of the subject.

The central database may be disposed inside or outside the psychological intervention cabin and connected by means of wireless and/or wired communications.

Before the arithmetic unit establishes the multi-dimensional state point of the subject according to the mathematical model, the arithmetic unit may check the integrity of the acquired data of the subject required by each dimension first. If the integrity reaches the preset standard, the arithmetic unit establishes the multi-dimensional state point of the subject and matches it; if the integrity does not reach the preset standard, it needs to return to the data acquisition module to continue to acquire relevant missing information until the integrity reaches the preset standard.

After determining that the subject is in a depressed state, the arithmetic unit in the data processing module calls the intervention plan corresponding to the matching multi-dimensional state point in the central database, and guides the intervention module to perform the intervention procedure on the subject according to the corresponding intervention plan. In the intervention procedure, the data acquisition module continuously acquires information of the subject and continuously transmits the acquired information to the data processing module. The arithmetic unit of the data processing module forms a new multi-dimensional state point of the subject based on the received data, searches for a new multi-dimensional state point matching the subject in the central database, matches it for the corresponding new intervention procedure, and guides the intervention module to adjust the intervention procedure. Iteratively, the intervention procedure is continuously adjusted during the intervention process to find the intervention plan most suitable for the subject.

Further, the data processing module also includes a memory unit. The data acquired by the data acquisition module can be first transmitted to the memory unit. The arithmetic unit receives the data from the memory unit for analysis and operation.

Wherein, during the intervention procedure, the intervention procedure of the intervention module and the data acquisition operation of the data acquisition module are performed alternately. That is, after the intervention module has intervened for a period, the data acquisition module acquires the subject's relevant information. The data processing module receives the acquired information of the data acquisition module, generates a new multi-dimensional state point based on the acquired information, matches it, and further readjusts the intervention procedure. Iteratively, the optimal intervention procedure are achieved during continuous adjustments. The system can adjust the intervention procedure during the intervention process according to the specific conditions of different subjects, thereby providing targeted psychological counseling, assessment and intervention procedure, and improving the success rate of psychological assessment and intervention.

Further, the data acquisition module includes one or more of the following data acquisition units: a background data acquisition unit, a physiological indicator acquisition unit, an audio acquisition unit, a video acquisition unit, a physical variable acquisition unit, and a chemical acquisition unit. The background data acquisition unit acquires the background data of the subject in the form of a questionnaire survey. The background data includes the subject's basic information, social relationships, and interests and hobbies. The basic information includes height, weight, and occupation. The social relationships include one or more relations of family members, relatives, and friends.

The physiological indicator acquisition unit includes a number of contact and/or non-contact physiological indicator acquisition devices. The acquired physiological indicators include heart rate, blood pressure, brain waves, respiratory rate, blood oxygen, electrocardiogram, mental and physical stress indicators based on heart rate, autonomic nervous system measurement (sympathetic and parasympathetic) and/or vascular aging, etc.

The audio unit includes a number of audio sensors distributed in the housing. The audio sensors are responsible for acquiring the verbal, non-verbal information of the subject and/or ambient noise in the housing. The non-verbal information of the subject includes the sounds made through body movements.

The video acquisition unit includes a number of video sensors distributed in the housing. The video sensors are responsible for acquiring the facial expressions and/or body movements of the subject. The combination of several video sensors in the video acquisition unit is used to calculate the location where the subject's body and/or body movements occur in the three-dimensional space. The acquired facial expressions include facial expressions the subject makes consciously and unintentional and unnoticeable micro-expressions. The micro-expressions last shorter and can better reflect people's true feelings and motivations. The micro-expressions in psychology tend to refer to those suppressed expressions.

The physical variable acquisition unit includes pressure sensors, and/or an acceleration sensor, and/or a gravity sensor, used to acquire the force generated during the subject's physical movements.

The chemical unit includes a number of chemical sensors. The chemical sensors are used to acquire the smell of the subject and the ambient temperature and humidity in the housing.

The intervention module is divided into a cross-border zone, a deep communication zone, and an empowerment zone according to functional zones. However, this division of functional zones is only to illustrate that the intervention module has the above-mentioned functions, not to limit the spatial positional relationship of the above-mentioned functional modules. That is, the spatial positional relationship between the functional modules may be separated or partially overlapped, or all overlapped.

The cross-border zone uses the data information acquired by the data acquisition module to personalize the arrangement of the environment in the cabin housing. The personalized arrangement includes the arrangement of one or more parameters, such as the ambient temperature and humidity, brightness, music, odor, and vibration in the housing to help the patient relax and generate a sense of trust so that the subject can walk into a relatively relaxed virtual environment from the real society, unpack his unintentional protection and packaging, and express more of the things that cannot be expressed in his inner world. The deep communication zone is the main body of the intervention module. The deep communication zone adjusts the intervention procedure by continuously intervening with the subject and acquiring the subjects' indicator parameters during the intervention process, finding the most suitable intervention procedure for the subject, and finally achieving the purpose of transitioning the subject from a depressed state to a normal state. After the intervention procedure of the deep communication zone is completed, the subject enters the empowerment zone. The empowerment zone can provide a place for the subject to rest, such as a recliner for the subject to rest. The empowerment zone empowers the subject. The subject exits the cabin after the empowerment. The empowerment operation is mainly to empower the subject using acoustic, optical, electrical, chemical, and/or magnetic methods. During the empowerment operation, the data acquisition module continuously monitors the physiological data of the subject and forms a multi-dimensional state point of the subject at the time. The arithmetic unit of the data processing module finds the multi-dimensional state point matching the point in the central database, determines that the subject is in a depressed state, a normal state, or an excited state at the time, and records the determination result in the memory unit of the data processing module for archiving so that the determination result can continue to be used as background data when the subject comes again next time.

Furthermore, the psychological intervention cabin provided by the present invention requires learning and training before practical application. The learning and training refer to that the psychological intervention cabin needs to learn different response actions of counselors made to different emotional states through learning a large number of sample cases of intervention plans of different subjects. A test is carried out after learning sufficient sample cases. When testing, for different subjects, the response plan of the data processing module and the intervention module in the psychological intervention cabin is compared to the response plan of the counselor in this state to determine if it is the same. If the same, the learning is completed and the test is passed. If not the same, the psychological intervention cabin returns to learning until the test is passed. After passing the test, the psychological intervention cabin can be applied to use. The psychological intervention cabin put into use has a large number of processing plans for dealing with different emotional states, which are stored in the central database.

In addition, the psychological intervention cabin provided by the present invention also has the functions of regular updating and re-learning. Specifically, after a period of psychological assessment and intervention, it needs to learn and test new cases, constantly adding new data, better assessment and intervention methods.

The present invention also provides a psychological assessment and intervention method, which is used to find an intervention procedure suitable for the subject, and continuously adjusts the intervention procedure to achieve the purpose of optimization during the intervention process, including the following steps:

Step 1: Acquire background data of each individual in a target group, including various physiological indicator parameters and/or psychological indicator parameters; establish a multi-dimensional coordinate system, each dimension of the coordinate system represents a type of indicator parameter; depict the multi-dimensional state point of each individual in the target group according to the indicator parameters of each individual in the target group, wherein all the multi-dimensional state points form a state probability distribution diagram which is stored in the central database;

Step 2: Calculate at least one growth curve between the multi-dimensional state point and the ideal state point of each individual in the target group. The calculation method is: successively searching around for the state of the largest probability distribution in the same step length from the initial state point to the ideal state point, and gradually extending and developing along the direction of the state point of the largest probability distribution, obtaining at least one path, the final position of which is in the ideal state region;

The relationship between the ideal state point and the ideal state region is: all the region that fluctuates around the ideal state point within a certain range is the ideal state region. As long as the final position is in the ideal state region, it can be considered that the end of the growth curve reaches an ideal state;

Step 3: Sort the at least one path obtained by the above calculation and select the optimal path therefrom. The sorting and selection criteria is that the shorter the total length of the path, the better the path.

Step 4: After a subject enters the psychological intervention cabin, the data acquisition module acquires the relevant indicators of the subject. The arithmetic unit in the data processing module reads the relevant indicators, forms a multi-dimensional initial state point of the subject, and matches the multi-dimensional initial state point to the multi-dimensional state points in the state probability distribution diagram in the central database to find the closest matching point;

Step 5: Find the optimal path from the matching point to the ideal state region according to the matched matching point. The optimal path is used to guide the psychological intervention procedure of the subject;

Step 6: Call out the corresponding physiological indicator parameters according to a number of state points on the optimal path; sequentially search for the intervention direction from the previous state point of physiological indicator parameter to the next state point of physiological indicator parameter starting from the physiological indicator parameter corresponding to the initial state point; and implement the relevant actions specified in the corresponding intervention procedure;

Step 7: After intervening with the subject for a period At according to the psychological intervention procedure corresponding to the optimal path, the data acquisition module acquires data from the subject again, and compares the data acquired this time to the data corresponding to the corresponding standard state point on the optimal path. If the deviation of the two sets of data is within a threshold range, continue on the intervention path of step 6, if the deviation of the two sets of data exceeds the threshold, proceed to step 8;

Step 8: Calculate the distance from the deviated state point to the ideal state point and the distance from the deviated state point back to the expected state point until the ideal state point; compare the length of the two; choose the shortest path to continue with the intervention; and repeat this method until reaching the ideal state point.

Further, in step 4, after the data acquisition module acquires the indicators of the subject, the arithmetic unit of the data processing module checks the integrity of the acquired data of the subject required by each dimension first. If the integrity reaches the preset standard, the arithmetic unit establishes the multi-dimensional state point of the subject and matches the multi-dimensional state point of the subject; if the integrity does not reach the preset standard, it needs to return to the data acquisition module and continue to acquire relevant missing information until the integrity reaches the preset standard.

Moreover, the psychological assessment and intervention method also includes step 9. After the intervention procedure of step 8 is completed, the subject needs to enter the empowerment zone for an empowerment operation. The empowerment operation is performed by acoustic, optical, electrical, chemical, and/or magnetic methods to empower the subject; wherein, the acoustic method mainly includes playing some encouraging words, playing music or special sound wave signals through microphones to effectively intervene with the subject's subconscious mind; the optical method includes embedding a specific pattern in an image or illuminate pattern to subconsciously hypnotize or induce the subject; the electromagnetic method includes transcranial electrical stimulation and transcranial magnetic stimulation, which are mainly sticking electrodes or electromagnetic units on the head in a contact or non-contact manner, and sending modulated electrical or magnetic signals to the brain, so that these modulated signals affect the functioning of the cerebral cortex and affect the consciousness or mental state of the subject; the chemical method includes spreading or spraying a certain type of chemical molecules to make the brain generate special sensations, or induce and solidify a certain type of specific memories, taking advantage of the special position of the olfactory bulb and olfactory nerve in the brain to optimize, strengthen or even achieve the effects that other intervention methods cannot achieve. If the released chemical molecules are neurotransmitters (exogenous or endogenous), these chemical molecules can participate in the discharge conduction function of neurons and directly change the thinking mode of the brain.

In the intervention process, it is required to continuously acquire the physiological indicator parameters of the subject, compare the acquired real-time physiological indicator parameters to the ideal physiological indicator parameters of the intervention stage, and determine whether there is an offset. If there is no offset, continue to intervene on the original path. If an offset occurs, reassess using the calculation method in step 8 to generate a new intervention path. This adjustment method lasts until the end of the entire psychological intervention procedure. Through this method of continuously adjusting the intervention procedure, it is possible to select the optimal plan suitable for different individuals, thereby solving the problem efficiently.

The beneficial effects of the present invention are:

1) The establishment of an independent psychological intervention cabin, combined with artificial intelligence, big data intervention, and other technologies, achieves that machines replace psychological counselors, which has alleviated the shortage of psychological counselors to a certain extent;

2) In addition, the independent psychological intervention cabin achieves that subjects can receive psychological assessment and intervention services alone, which does not need the intervention of others and also improves personal privacy;

3) For different subjects, taking advantage of big data processing technology, scientific sensor analysis instruments are used to comprehensively and accurately measure the user's psychophysiological indicators to obtain objective data basis and measurement standards based on big data. In addition, the system can also provide different psychological intervention procedures for different subjects, and adjust the intervention procedure in time according to the subject's dynamics in the intervention process when targeting the same subject, rationalizing and optimizing the procedure to achieve the best intervention effect;

4) In addition to the above-mentioned beneficial effects, the system provided by the present invention can provide services at any time, compared to the fact that subjects need to queue for an appointment and coordinate time for real counselors, and also can avoid the disadvantage that there may exist inconsistent information for each intervention data and result when facing different real people, in the case that multiple psychological interventions are required. The system of the present invention can ensure the consistency of services and reduce service costs to a certain extent.

### BRIEF DESCRIPTION OF THE DRAWINGS

In order to more clearly describe the technical solutions in the specific embodiments of the present invention, the following will briefly introduce the drawings needed in the description of the embodiments. Apparently, the drawings described below are only some embodiments of the present invention. For one of ordinary skill in the art, other drawings can be obtained based on these drawings without creative work.

FIG. 1 is a schematic diagram of the data flow between the modules of an intelligent psychological assessment and intervention system based on an independent space provided in the first embodiment of the present invention.

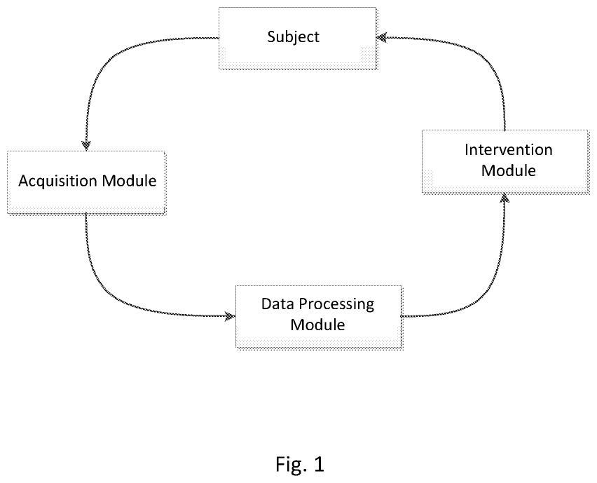

FIG. 2 is a detailed structure diagram of each module of an intelligent psychological assessment and intervention system based on an independent space provided by the fourth embodiment of the present invention and a schematic diagram of the data flow between the modules.

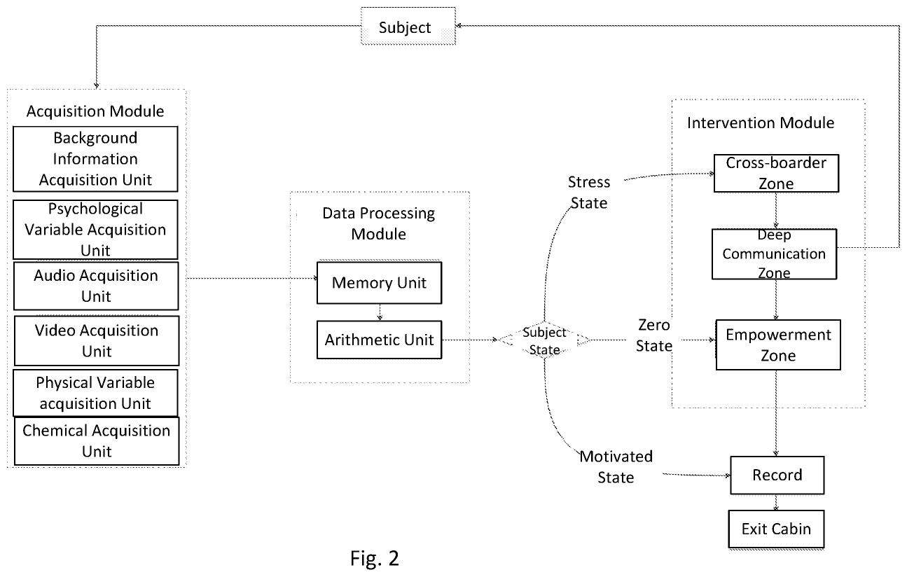

FIG. 3 is a schematic diagram of learning and testing of an intelligent psychological assessment and intervention system based on an independent space provided by the fifth embodiment of the present invention before leaving the factory.

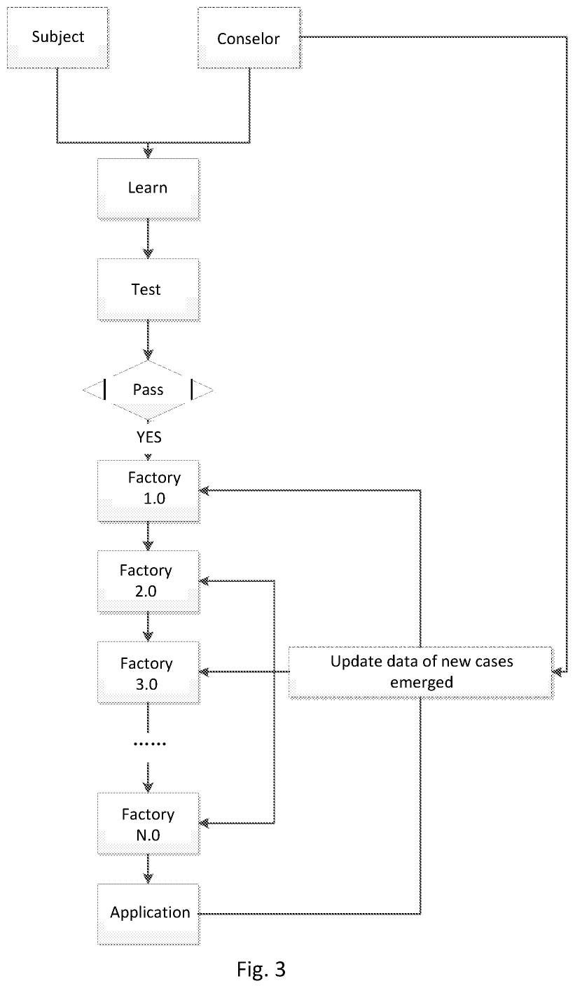

FIG. 4 is a flowchart of an intelligent psychological assessment and intervention method based on an independent space provided by the seventh embodiment of the present invention.

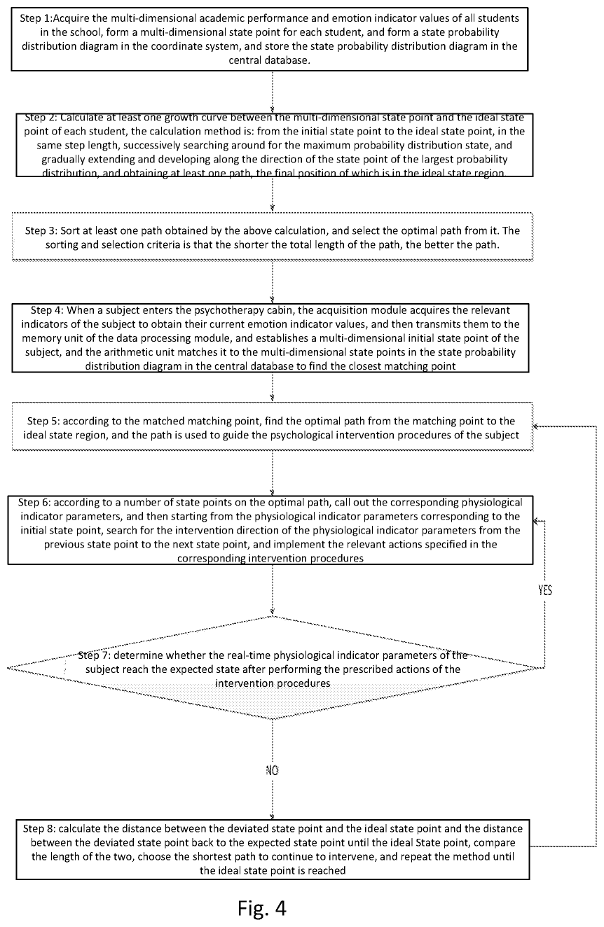

### DETAILED DESCRIPTION

The technical solutions in the specific embodiments of the present invention will be described clearly and completely below. Obviously, the described embodiments are only a part of the embodiments of the present invention, rather than all the embodiments. Based on the embodiments of the present invention, all other embodiments obtained by one of ordinary skill in the art without creative work shall fall within the scope of the present invention.

### Embodiment 1

The first embodiment provides an intelligent psychological assessment and intervention system based on an independent space, including a psychological intervention cabin and a central database capable of achieving data interaction with it, wherein the psychological intervention cabin is an independent space which is independently set up and has a confined space. The psychological intervention cabin includes a housing, a data acquisition module disposed in the housing, an internal or external data processing module and an intervention module.

The data processing module is connected to the central database, the data acquisition module and the intervention module by means of wireless and/or wired communications. The central database stores a plurality of multi-dimensional state points of different individuals, and intervention plans for different individuals. The individuals' multi-dimensional state points refer to selecting multiple different indicator parameters and establishing a multi-dimensional coordinate system first, and depicting the multi-dimensional state point corresponding to each individual in the multi-dimensional coordinate system according to the indicator values corresponding to each individual, thereby forming a model database.

The data acquisition module is responsible for acquiring ambient information in the housing of the psychological intervention cabin and relevant information of the subject. The ambient information in the housing includes ambient temperature and humidity, and ambient noise. The relevant information of the subject includes heart rate, blood pressure, blood oxygen, electrocardiogram, breathing rate, languages, body movements, expressions and smells, etc.

The data processing module in this embodiment includes a memory unit and a arithmetic unit. The memory unit receives all information of the subject acquired by the data acquisition module and the ambient information in the housing. The arithmetic unit reads data from the memory unit, fuses all the received data according to the mathematical model, and establishes a multi-dimensional state point of the subject in the multi-dimensional space coordinate system. The arithmetic unit matches the multi-dimensional state point of the subject to the multi-dimensional state points in the central database, finds the point that is the same or similar to the subject's multi-dimensional state point, and determines whether the subject's multi-dimensional state point is in a depressed, normal or excited state. In the case it is in a depressed state, the arithmetic unit instructs the intervention module to intervene with the subject according to the intervention procedure at this point. If it is in a normal state, the subject is empowered; if it is in an excited state, the subject is prompted to leave the cabin immediately. The depressed state, normal state and excited state described herein refer to judging whether the subject is in a negative state, a peaceful state or a positive state based on the multi-dimensional data of the subject. That is, it is required to intervene with the subject to achieve the purpose of adjusting the state of mind when the subject is in a negative state; it is required to perform empowerment to achieve a positive state of mind when the subject is in a peaceful state of mind; when the subject is in a positive state of mind, it means that the subject is currently in a mentally healthy state and no psychological intervention is required for the time being.

In addition, the central database in this embodiment may be disposed inside the psychological intervention cabin, or outside the psychological intervention cabin. The central database may be in wireless or wired communications with the modules in the psychological intervention cabin, as long as data interaction between the central database and each module can be achieved.

The mathematical model fuses all the acquired information received to establish a multi-dimensional state point cloud of the subject. The mathematical model refers to the establishment of a multi-dimensional spatial coordinate system. The coordinate of each dimension of the spatial coordinate system is used to characterize an indicator value of the subject. The indicator values of multiple dimensions establish a multi-dimensional state point of the subject at a specific moment. Multiple multi-dimensional state points in the central database are also established through the same multi-dimensional coordinate system, and a state point probability distribution diagram is formed under the multi-dimensional coordinate system.

In this embodiment, after the arithmetic unit establishes the multi-dimensional state point of the subject according to the mathematical model, it is required to check the integrity of each dimensional data of the multi-dimensional state point first, that is, check whether the data of each dimension of the point has been acquired. The data integrity standard in this embodiment is 85% and above. That is, if the data integrity in the multi-dimensional coordinate system of the subject reaches 85% or above, the arithmetic unit matches the subject's multi-dimensional state point and finds the same or similar point to it. If the integrity does not meet the preset standard, it is required to return to the data acquisition module to continue to acquire relevant missing information until the integrity reaches the preset standard.

After determining that the subject is in a depressed state, the arithmetic unit in the data processing module calls the intervention plan corresponding to the matched multi-dimensional state point in the central database, and guides the intervention module to perform the intervention procedure on the subject according to the corresponding intervention plan. In the intervention procedure, the data acquisition module continuously acquires information of the subject, and continuously transmits the acquired information to the memory unit in the data processing module. The arithmetic unit of the data processing module forms a new multi-dimensional state point of the subject according to the received data in the memory unit, finds a new multi-dimensional state point that matches the subject in the central database and matches it with the corresponding new intervention procedure, and guides the intervention module to adjust the intervention procedure, repeating this cycle to continuously adjust the intervention procedure during the intervention process and seek the intervention plan most suitable for the subject.

In the intervention process, the intervention procedure of the intervention module and the information acquisition operation of the data acquisition module are performed alternately. That is, after the intervention module has performed intervention for a period, the data acquisition module acquires the relevant information of the subject. The data processing module receives the acquired information of the data acquisition module, and generates a new multi-dimensional state point according to the acquired information, matches it, and further readjusts the intervention procedure. By repeating this cycle, the intervention procedure is continuously adjusted to achieve optimization. The psychological intervention system provided in this embodiment can adjust the intervention procedure during the intervention process according to the particular conditions of different subjects, thereby providing targeted psychological counseling, assessment and intervention procedure, and improving the success rate of psychological assessment and intervention.

### Embodiment 2

The second embodiment is based on the first embodiment. The added technical features are that the data acquisition module in this embodiment includes a background data acquisition unit, a physiological indicator acquisition unit, an audio acquisition unit, a video acquisition unit, a physical variable acquisition unit, and a chemical acquisition unit.

The background data acquisition unit acquires background data of the subject in the form of a questionnaire survey. The questionnaire survey form may employ a paper questionnaire survey form and/or an electronic device survey form. When a paper questionnaire survey form is employed, the statistical survey data can be entered through image acquisition and/or manually after the questionnaire survey is completed. The electronic device survey form can include manual operation and/or voice questions and answers. The acquired background data includes the subject's basic information, social relationships, hobbies, etc. The basic information includes height, weight, occupation, etc. The social relationships include family member relations, relatives relations, friends relations, etc.

The physiological indicator acquisition unit includes contact and/or non-contact physiological indicator acquisition devices. The acquired physiological indicators include heart rate, blood pressure, brain waves, respiration frequency, blood oxygen, electrocardiogram, etc., wherein the contact physiological indicator acquisition devices include heart rate acquisition devices, blood pressure and blood oxygen monitors, bracelets, brain wave detectors, myoelectric skin sensors, and near-infrared brain function detection devices. The non-contact physiological indicator acquisition devices include nuclear magnetic resonance detectors, heart magnetic and brain magnetic measuring instruments, doppler radar sensors, and infrared temperature sensors.

The audio acquisition unit includes a number of audio sensors distributed in the housing. The audio sensors are responsible for acquiring the verbal and non-verbal information of the subject and the ambient noise in the housing. The verbal information of the subject is the content of the speech, and the non-verbal information includes the sounds the subject makes through body movements. Specifically, the number of audio sensors in this embodiment are multiple microphones distributed in the housing of the psychological intervention cabin, which simultaneously acquire the verbal information expressed by the subject and the sounds the subject makes by various body movements in the cabin, such as the sounds of stomping, pounding tables and chairs, etc. The number of audio sensors can also monitor the ambient noise in the housing at the same time. When the ambient noise in the housing exceeds a certain decibel, the subject can be reminded that it is not suitable to perform psychological intervention this time and it is recommended to do it next time.

The video acquisition unit includes a number of video sensors distributed in the housing. The video sensors are responsible for acquiring the facial expressions and body movements of the subject. The combination of the number of video sensors in the video acquisition unit is used to calculate the location where the subject's body and/or limb movements occur in the three-dimensional space. Specifically, the number of video sensors in this embodiment include multiple cameras distributed in the housing of the psychological intervention cabin. The cameras can acquire changes in facial expressions of the subject, and body movements. Multiple cameras may be employed in different directions to acquire and locate the specific coordinate position of the subject's body movements. The facial expressions described in this embodiment include the obvious facial expressions the subject makes consciously as well as unintentional and unnoticeable micro-expressions. The micro-expressions have a short duration and small amplitude and are not easily detectable, but such micro-expressions can better reflect people's true feelings and motivations. The micro-expressions in psychology tend to refer to those expressions that are suppressed. By acquiring these micro-expressions, the true psychological feelings of the subject can be better judged. The location of the specific coordinate position of the subject's body movements can better reflect the psychological state of the subject. For example, people who are physically and mentally exhausted will spread out in a chair, or lie on the table when they are under stress, or their legs will involuntarily shake and their mouth or throat will swallow saliva when they are nervous. Through the specific position of these body languages, the current psychological state of the subject can be calculated.

The physical variable acquisition unit includes pressure sensors and/or an acceleration sensor and/or a gravity sensor, used to acquire the force generated by the subject's body movements. Specifically, the force acquired in this embodiment includes at least one of the force of the subject hitting the object, the strength of stomping, the frequency of repetitive force (that it is a continuous rapid tap or a long interval of gravity taps), and acceleration indicators, which is used to evaluate the extent of the mood swings or emotional intensity of the subject at the time.

The chemical unit includes a number of chemical sensors, which are used to acquire the smell of the subject and the ambient temperature and humidity in the housing. Specifically, the chemical sensors in this embodiment acquire the smell of the subject, which may be the smell of the subject's body and/or the smell of the chemical molecules emitted by the body itself, which are used to judge the preference of the subject.

### Embodiment 3

This embodiment is based on the second embodiment. The added technical features are that after the arithmetic unit in the data processing module extracts the acquired information of the subject, the arithmetic unit evaluates the integrity of the above-mentioned data first. If the integrity reaches the preset standard, the arithmetic unit establishes a multi-dimensional state point of the subject and then matches the point to the multiple multi-dimensional state points in the central database. If the integrity does not meet the preset standard, it is required to return to the data acquisition module to continue to acquire relevant missing information about the subject until the integrity reaches the preset standard.

The data integrity standard in this embodiment is 85% and above. Alternatively, the standard can also be modified. The higher the standard, the more accurate the evaluation will be.

### Embodiment 4

This embodiment is based on the third embodiment. The additional technical features include that the intervention module can be divided into a cross-border zone, a deep communication zone, and an empowerment zone according to functions. But this division of functional zones is only for illustration that there exist the above-mentioned functional modules in the intervention module, not intended to limit the spatial positional relationship between the above-mentioned several functional modules. That is, the spatial positional relationship between the above-mentioned functional modules may be separated, partially overlapped, or all overlapped.

The cross-border zone uses the data information acquired by the data acquisition module to personalize the arrangement of the environment in the cabin housing. Specifically, the personalized arrangement includes acquiring ambient temperature and humidity in the housing to adjust the temperature and humidity in the housing, thereby achieving the purpose of making people comfortable; and using original sensory technologies, such as immersive projection technology, color, smell, music, voice, etc., and employing technical means of sound and light to create a subject comfortable and relaxing environment. The specific technical means are: adjusting the brightness in the housing, playing the music that the subject likes, releasing the smell that the subject likes, and generating a relaxing vibration frequency, finally relaxing the patient and generating a sense of trust, so that the subject can walk into a relatively relaxed virtual environment from the real world to let him remove his unintentional protection or packaging, and express more of the things that cannot be expressed in his inner world. The personalized arrangement is a dynamic adjustment process, that is, an initial personalized arrangement is first performed based on the subject's background data. During this process, the data acquisition module continues monitoring the subject. If it is monitored that the initial personalized arrangement can enable the development of the subject's psychophysiological indicators towards a positive direction, it indicates that the direction of the personalized arrangement is correct. If the indicators develop towards a negative direction, the direction of the personalized arrangement needs to be adjusted. This cycle repeats until achieving the optimal personalized arrangement.

The deep communication zone is the main body of the intervention module. It adjusts the intervention procedure by continuously intervening with the subject and acquiring indicator parameters during the intervention process for the subject, finds the most suitable intervention procedure for the subject, and finally achieves the purpose of transitioning the subject from a depressed state to a normal state. The specific process is the same as the first embodiment.

After the intervention procedure of the deep communication zone is completed, the subject enters the empowerment zone. The empowerment zone can provide a place for the subject to rest, such as a recliner, sofa, or soft cushion, etc., and empowers the subject. After the empowerment, the subject exits the cabin. The empowerment operation empowers the subject through acoustic, optical, electrical, chemical and/or magnetic methods. For example, the acoustic, optical, electrical, chemical, and/or magnetic methods include: the acoustic method mainly includes playing some encouraging words, playing music or special sound wave signals using a microphone to effectively intervene with the subject's subconscious; the optical method includes embedding a specific pattern in an image or illuminate pattern to subconsciously hypnotize or induce the subject; the electromagnetic method includes transcranial electrical stimulation and transcranial magnetic stimulation, which are mainly sticking electrodes or electromagnetic units to the head in a contact or non-contact manner and sending modulated electrical or magnetic signals to the brain, so that these modulated signals affect the functioning of the cerebral cortex, thereby affecting the consciousness or mental state of the subject; the chemical method includes spreading or spraying a certain type of chemical molecules to make the brain generate special sensations, or induce and solidify a certain type of specific memories, taking advantage of the special position of the olfactory bulb and the olfactory nerve in the brain to optimize, strengthen and even achieve the effect that other intervention methods cannot achieve; if the released chemical molecules are neurotransmitters (exogenous or endogenous), these chemical molecules can participate in the discharge conduction function of neurons, thereby changing the thinking mode of the brain more directly.

In the empowerment operation, the data acquisition module continuously monitors the physiological data of the subject to form the multi-dimensional state point of the subject at the time. The arithmetic unit of the data processing module finds the multi-dimensional state point matching it in the central database, determines that the subject is in a depressed state, normal state or excited state, and records the determination result in the memory unit in the data processing module for archiving, which will be continually used as background data when the subject comes again next time.

### Embodiment 5

The difference between this embodiment and the foregoing embodiments is that the psychological intervention cabin provided in this embodiment requires learning and training before practical application. The learning and training refer to that the psychological intervention cabin needs to learn different response actions of the psychologist made to different emotional states through learning a large number of sample cases of different subjects' intervention plans. A test is carried out after the psychological intervention cabin learns sufficient sample cases. When testing, for different subjects, the response plan of the data processing module and intervention module in the psychological intervention cabin is compared to the response plan of the psychological counselor in this state to determine if it is the same. If the same, the learning is completed and the test is passed. If not the same, it is required to return to learning until the test is passed. After passing the test, the psychological intervention cabin can be used, and the psychological intervention cabin put into use has a large number of processing plans for dealing with different emotional states, which are stored in the central database.

In the learning and training described in this embodiment, a large number of sample cases of intervention plans of different subjects are learned, which can be on-site teaching or recorded video teaching. Specifically, the on-site teaching refers to the setup of a psychological intervention cabin on the scene at the same time while the psychological counselor is conducting psychological assessment and intervention for different subjects. The psychological intervention cabin learns the processing plans of the psychological counselor facing different subjects. The recorded video teaching means that videos can be recorded in advance as teaching cases when the psychological counselor conducts psychological assessment and intervention on the subject. The psychological intervention cabin can learn and train at a later stage by learning the recorded teaching cases. Similarly, the comparison between the response plans of the psychological intervention cabin and the psychological counselor during the testing stage may be a direct comparison test when the psychological counselor and the psychological intervention cabin are present at the same time, or conducted through pre-recorded videos to perform the comparison test.

### Embodiment 6

On the basis of the above-mentioned embodiment, this embodiment includes additional technical features that the psychological intervention cabin in this embodiment further has the functions of regular updating and re-learning.

Specifically, after a period of psychological assessment and intervention, supplementary learning and testing of new cases are required. In addition, learning and testing are also required for better psychological assessment methods that emerge and better intervention methods for existing cases, thereby achieving the purpose of constantly supplementing new data and better assessment and intervention methods. The method of relearning and testing is the same as the learning and testing before the psychological intervention cabin is put into use in the fourth embodiment, which will not be repeatedly described here.

### Embodiment 7

This embodiment provides a method of psychological assessment and intervention on the basis of the psychological intervention cabin described in the above-mentioned embodiments 1 to 6. The method is used to find a suitable intervention procedure for the subject, and continuously adjusts the intervention procedure during the intervention process to achieve the purpose of optimization.

In this embodiment, taking the school as the background, the psychological intervention cabin is set up in the school area, using the student's academic performance and emotion indicators as the criteria for determining whether the psychological intervention process is successful. The ideal state is that the academic performance is good and the emotion indicators are healthy. For the convenience of description in this embodiment, the employed X-axis coordinate in the multi-dimensional coordinate system is the emotion indicator, and the other-dimensional coordinates are each academic performance. The psychological assessment and intervention method includes the following steps:

Step 1: Acquire background data of each individual in a target group, including various physiological indicator parameters and/or psychological indicator parameters; establish a multi-dimensional coordinate system, each dimension of the coordinate system represents a type of indicator parameter; depict the multi-dimensional state point of each individual of the target group according to the indicator parameters of each individual in the target group, all the above-mentioned multi-dimensional state points form a state probability distribution diagram, the state probability distribution diagram is stored in the central database;

In this embodiment, the multiple academic performances and emotion indicator values of all students in the school are acquired to form the multi-dimensional state point of each student. The multi-dimensional state point of each student is depicted in the coordinate system, finally forming a state probability distribution diagram. The state probability distribution diagram is stored in the central database.

Specifically, the multi-dimensional academic performances acquired in this step may include the academic performance of multiple courses. The emotion indicator values may be obtained by the teacher and/or parents in the form of questionnaire scoring. The coordinate system is established by the student's multiple academic performances and emotion indicator values. The multi-dimensional state point formed for each student is depicted in the established coordinate system to form a probability state distribution diagram based on the acquired background data.

Step 2: Calculate at least one growth curve between each student's multi-dimensional state point and the ideal state point. The calculation method is: successively searching for the state of the largest probability distribution around in the same step length from the initial state point to the ideal state point, and gradually extending and developing along the direction of the state point of the largest probability distribution, to obtain at least one path, the final position of which is in the ideal state region;

Wherein, the ideal state point may be one of the points in the ideal state region. That is, due to the probability distribution, all the region within a certain threshold range near the most ideal state point is referred to as the ideal state region, and all the points located in the region can be referred to as ideal state points.

Step 3: Sort the at least one path obtained by the above calculation, and select the optimal path therefrom. The sorting and selection criteria is that the shorter the total length of the path, the better the path;

Step 4: After a subject enters the psychological intervention cabin, the data acquisition module acquires the subject's relevant indicators to obtain his/her current emotion indicator values; transmits them to the memory unit in the data processing module to establish a multi-dimensional initial state point of the subject. The arithmetic unit matches it to the multi-dimensional state points in the state probability distribution diagram in the central database and finds the closest matching point;

Considering that as a student, his/her academic performances fluctuate little in a short term, the multi-dimensional initial state point of the subject is established based on the corresponding original academic performances in the central database and the currently acquired emotion indicator values;

Step 5: Find the optimal path from the matching point to the ideal state region according to the matched matching point. The path is used to guide the psychological intervention procedure of the subject;

Step 6: Call out the corresponding physiological indicator parameters according to a number of state points on the optimal path, and then sequentially search for the intervention direction from the previous state point of physiological indicator parameter to the next state point of physiological indicator parameter starting from the physiological indicator parameter corresponding to the initial state point, and implement the relevant actions specified in the corresponding intervention procedure;

Step 7: After intervening with the subject for a period At according to the psychological intervention procedure corresponding to the optimal path, the data acquisition module acquires data from the subject again, and compares the data acquired at the time to the data corresponding to the corresponding standard state point. If the comparison of the two sets of data is within a threshold range, continue on the intervention path of step 6, if the deviation of the two sets of data exceeds the threshold, proceed to step 8;

Step 8: Calculate the distance from the deviated state point to the ideal state point and the distance from the deviated state point back to the expected state point until the ideal state point, compare the length of the two distances, choose the shortest path to continue the intervention, and repeat the method until reaching the ideal state point.

In the intervention process, it is required to continuously acquire physiological indicator parameters of the subject, compare the acquired real-time physiological indicator parameters with the ideal physiological indicator parameters at the intervention stage, and determine if a deviation occurs. If a deviation does not occur, continue to intervene on the original path; if a deviation occurs, re-assess using the calculation method in step 8 above to generate a new intervention path. This adjustment method lasts until the end of the entire psychological intervention process. Through this method of continuously adjusting the intervention process, it is possible to select the best solution suitable for each individual for different subjects, thereby solving the problem efficiently.

And in step 4, after the data acquisition module acquires the indicators of the subject, the arithmetic unit of the data processing module checks the integrity of the acquired data of the subject required by each dimension first. If the integrity reaches the preset standard, the arithmetic unit establishes a multi-dimensional state point about the subject, and matches the multi-dimensional state point of the subject; if the integrity does not reach the preset standard, it is required to return to the data acquisition module to continue to acquire relevant missing information until the integrity reaches the preset standard.

In addition, after the intervention procedure of step 8 above is completed, the subject needs to enter the empowerment zone for an empowerment operation. The empowerment operation empowers the subject through acoustic, optical, electrical, chemical and/or magnetic methods. Wherein, the acoustic method mainly includes playing some encouraging words, playing music or special sound wave signals through microphones to effectively intervene with the subject's subconscious; the optical method includes embedding a specific pattern in an image or illuminate pattern, subconsciously hypnotizing or inducing the subject; the electromagnetic method includes transcranial electrical stimulation and transcranial magnetic stimulation, which are mainly sticking electrodes or electromagnetic units on the head in a contact or non-contact manner, and sending modulated electrical or magnetic signals to the brain to allow these modulated signals affect the functioning of the cerebral cortex, and affect the consciousness or mental state of the subject; the chemical method includes spreading or spraying a certain type of chemical molecules to make the brain generate special sensations, or induce and solidify a certain type of specific memories, taking advantage of the special position of the olfactory bulb and olfactory nerve in the brain to optimize, strengthen, and even achieve effects that cannot be achieved by other interventions. If the released chemical molecules are neurotransmitters (exogenous or endogenous), these chemical molecules may participate in the discharge conduction function of the neurons and directly change the thinking mode of the brain.

It is understandable that this embodiment only exemplarily describes the detailed principles of the psychological assessment and intervention method provided by the present invention taking a school as a background. The psychological intervention cabin provided by the present invention can also be used in other public areas, such as drug rehabilitation centers. In areas such as enterprises and institutions, they are different from schools in that they target different groups of people. Therefore, the parameter values selected when establishing a multi-dimensional coordinate system are different, but the basic processes used are the same.

The above provides a detailed introduction to the intelligent psychological assessment and intervention system and method based on an independent space provided by the present invention. Specific examples are used in this disclosure to illustrate the structure and working principle of the present invention. The description of the above embodiments is only for assisting to understand the method and core idea of the present invention. It should be pointed out that for those of ordinary skill in the art, without departing from the principle of the present invention, several improvements and modifications can be made to the present invention, and these improvements and modifications also fall within the scope of the claims of the present invention.

1. An intelligent psychological assessment and intervention system based on an independent space, characterized in that, the system comprises a psychological intervention cabin and a central database which achieves data interaction with the cabin;
the data processing module is connected to the central database, the data acquisition module, and the intervention module;
the psychological intervention cabin comprises a housing, a data acquisition module, an internal or external data processing module, and an intervention module;
the central database stores a plurality of sample individuals and corresponding intervention plans;
the data acquisition module acquires relevant physiological and psychological data of a subject and transmits it to the data processing module;
the data processing module fuses the data transmitted by the data acquisition module according to the mathematical model of the data processing module, matches the data with the samples in the central database, calls the intervention plan corresponding to the matched sample, and guides the intervention module to intervene with the subject according to the intervention plan.

2. The intelligent psychological assessment and intervention system based on an independent space of claim 1, characterized in that, the mathematical model of the data processing module is a multi-dimensional spatial coordinate system, the data processing module establishes the subject's multi-dimensional state point in the multi-dimensional spatial coordinate system based on the data acquired by the data acquisition module, matches it to multi-dimensional state points in the central database, and finds the same or similar matching point to it.

3. The intelligent psychological assessment and intervention system based on an independent space of claim 1, characterized in that, during the intervention process, the data acquisition module continuously acquires information of the subject and transmits the acquired information to the data processing module, the data processing module forms a new multi-dimensional state point of the subject based on the received data, matches it for a new corresponding intervention procedure in the central database, guides the intervention module to adjust the intervention procedure, iteratively, thereby continuously adjusting the intervention procedure during the intervention process and finding the most suitable intervention procedure for the subject.

4. The intelligent psychological assessment and intervention system based on an independent space of claim 1, characterized in that, the psychological intervention cabin is an independent space which is independently set up and has a confined space.

5. (canceled)

6. The intelligent psychological assessment and intervention system based on an independent space of claim 1, characterized in that, the data acquisition module also acquires ambient information in the psychological intervention cabin, including ambient temperature and humidity, and ambient noise.

7. The intelligent psychological assessment and intervention system based on an independent space of claim 2, characterized in that, the data processing module at least comprises an arithmetic unit, the matching process of the data processing module is implemented by the arithmetic unit; the arithmetic unit determines that the subject's multi-dimensional state point is in a depressed state, a normal state or an excited state after finding the point that is the same or similar to the subject's multi-dimensional state point in the case of a depressed state, the arithmetic unit guides the intervention module to intervene with the subject according to the intervention plan of the matched point in the case of a normal state, performs empowerment in the case of an excited state, prompts the subject to the cabin immediately.

8. (canceled)

9. The intelligent psychological assessment and intervention system based on an independent space of claim 3, characterized in that, the intervention procedure of the intervention module and the information acquisition operation of the data acquisition module are performed alternately, that is, the data acquisition module acquires relevant information of the subject after the intervention module intervenes for a period, then the data processing module receives the acquired information of the data acquisition module and generates a new multi-dimensional state point based on the acquired information, matches it, and further re-adjusts the intervention procedure; repeatedly, the intervention procedure is optimized through continuous adjustments.

10. The intelligent psychological assessment and intervention system based on an independent space of claim 1, characterized in that, the mathematical model refers to the establishment of a multi-dimensional spatial coordinate system, each spatial coordinate of the multi-dimensional spatial coordinate system represents an indicator value of the subject; the indicator value of multiple dimensions establish the multi-dimensional state point of the subject at a specific moment; the multiple multi-dimensional state points in the central database are also established by the same multi-dimensional coordinate system, and a state point probability distribution diagram is formed in the multi-dimensional coordinate system.

11. (canceled)

12. The intelligent psychological assessment and intervention system based on an independent space of claim 1, characterized in that, the data acquisition module includes one or more of the following data acquisition units: a background data acquisition unit, a physiological indicator acquisition unit, an audio acquisition unit, a video acquisition unit, a physical variable acquisition unit and a chemical acquisition unit.

13. The intelligent psychological assessment and intervention system based on an independent space of claim 12, characterized in that the background data acquisition unit acquires background data of the subject in the form of a questionnaire survey, the questionnaire survey is a paper questionnaire survey or an electronic device survey, the electronic device survey includes manual operation, and/or voice Q&A; the background data includes the subject's basic information, social relationships, interests and hobbies, the basic information includes height, weight, occupation, and the social relationships include one or more relations of family members, relatives, and friends; the physiological indicator acquisition unit includes a number of contact and/or non-contact physiological indicator acquisition devices, the acquired physiological indicators include heart rate, blood pressure, brain waves, respiratory rate, blood oxygen, electrocardiogram, heart rate-based mental and physical stress indicators, autonomic nervous system measurements, and/or vascular aging indicators; the audio unit includes a number of audio sensors distributed in the housing, the audio sensors are used to acquire the subject's verbal, non-verbal information and/or ambient noise in the housing; the video acquisition unit includes a number of video sensors distributed in the housing, the video sensors are responsible for acquiring the subject's facial expressions and/or body movements; the physical variable acquisition unit includes pressure sensors, and/or acceleration sensors, and/or gravity sensors, for acquiring the force generated by the subject's body movements; the chemical unit includes a number of chemical sensors, the chemical sensors are used to acquire the smell of the subject and ambient temperature and humidity in the housing.

14-16. (canceled)

17. The intelligent psychological assessment and intervention system based on an independent space of claim 13, characterized in that, the audio sensors are microphones, the acquired verbal information is the content of the subject's speech, and the non-verbal information includes the sounds the subject makes through body movements; the video sensors are cameras, the acquired facial expressions include expressions the subject makes consciously, and unintentional and unnoticeable micro-expressions; a combination of the number of video sensors in the video acquisition unit is used to calculate the location where the subject's physical and/or body movements occur in the 3D space.

18-22. (canceled)

23. The intelligent psychological assessment and intervention system based on an independent space of claim 1, characterized in that, the intervention module is divided into a cross-border zone, a deep communication zone and an empowerment zone according to functional zones; the cross-border zone uses the data information acquired by the data acquisition module to personalize arrangement of the environment in the cabin housing, the deep communication zone is the main body of the intervention module, adjusts the intervention procedure through continuous intervention with the subject and acquisition of the subject's indicator parameters in the intervention process, finds the most suitable intervention procedure for the subject, and finally achieves the purpose of transitioning the subject from a depressed state to a normal state, the subject enters the empowerment zone after the intervention procedure is completed, the empowerment zone provides a place for the subject to rest and empowers the subject, the subject leaves the cabin after the empowerment.

24. The intelligent psychological assessment and intervention system based on independent space of claim 23, characterized in that, the personalized arrangement includes the arrangement of one or more parameters including ambient temperature and humidity, brightness, music, smell, vibration, etc. in the housing, and the personalized arrangement is a dynamic adjustment process, that is, an initial personalized arrangement is first carried out according to the subject's background data, the data acquisition module continues monitoring the subject during the process, if it is monitored that the initial personalized arrangement makes the subject's psychophysiological indicators develop in a positive direction, it indicates that the direction of the personalized arrangement is correct, if the subject's psychophysiological indicators develop in a negative direction, the personalized arrangement is required to be adjusted, iteratively, until finding the optimal personalized arrangement.

25. An intelligent psychological assessment and intervention system based on independent space of claim 23, characterized in that, during the empowerment operation, the data acquisition module continuously monitors the physiological data of the subject, forming the subject's multi-dimensional state point at the time, the arithmetic unit of the data processing module finds the multi-dimensional state point matching the point in the central database, and determines that the subject is in a depressed state, normal state or excited state at the time, the determination result is recorded in the memory unit of the data processing module for archiving, so that it is used as background data when the subject comes again next time.

26. The intelligent psychological assessment and intervention system based on an independent space of claim 1, characterized in that, the data processing module further includes a memory unit, the memory unit receives relevant information of the subject and/or ambient information in the housing acquired by the data acquisition module, the arithmetic unit reads the data from the memory unit for processing.

27. The intelligent psychological assessment and intervention system based on an independent space of any of claim 1, characterized in that, the psychological intervention cabin requires learning and training before practical application, the learning and training refers to that the psychological intervention cabin needs to learn different response actions counselors make to different emotional states through learning a large number of sample cases of intervention plans of different subjects; after learning sufficient sample cases, a test is carried out, when testing, for different subjects, the response plan of the data processing module and the intervention module in the psychological intervention cabin is compared to the response plan of the counselor in this state to determine if it is the same: if the same, the learning is completed and the test is passed; if not the same, then it returns to learn until the test is passed.

28. The intelligent psychological evaluation and intervention system based on independent space of claim 27, characterized in that, the psychological intervention cabin also has the functions of regular updating and re-learning, after performing psychological assessment and intervention for a period, it is required to learn and test new cases, constantly adding new data and better assessment and intervention methods.

29. A psychological assessment and intervention method, characterized in that it comprises the following steps:
step 1: acquiring background data of each individual in a target group, including various physiological indicator parameters and/or psychological indicator parameters; establishing a multi-dimensional coordinate system, each dimension of the coordinate system represents a type of indicator parameter, depicting the multi-dimensional state point of each individual of the target group based on the indicator parameters of each individual in the target group, all the multi-dimensional state points form a state probability distribution diagram, and the state probability distribution diagram is stored in the central database;
step 2: calculating at least one growth curve between the multi-dimensional state point and the ideal state point of each individual in the target group, the calculation method is: from the initial state point to the ideal state point, in the same step length, successively searching for the state of the largest probability distribution around, and gradually extending and developing along the direction of the state point of the largest probability distribution to obtain at least one path, the final position of which is in an ideal state region; the relationship of the ideal state point and the ideal state region is: all the region that fluctuates around the ideal state point within a certain range is the ideal state region, as long as the final position is in the ideal state region, it is be considered that the end of the growth curve reaches the ideal state;
step 3: sorting the at least one path obtained by the above calculation, and selecting the optimal path therefrom, the sorting and selection criteria is that the shorter the total length of the path, the better the path;
step 4: After a subject enters the psychological intervention cabin, the data acquisition module acquires relevant indicators of the subject, the arithmetic unit in the data processing module reads the relevant indicators and forms a multi-dimensional initial state point of the subject, matches the multi-dimensional initial state point to the multi-dimensional state points in the state probability distribution diagram in the central database, and finds the closest matching point;
step 5: finding the optimal path from the matching point to the ideal state region according to the matched matching point, the path is used to guide the psychological intervention procedure of the subject;
step 6: according to a number of state points on the optimal path, calling out the corresponding physiological indicator parameters, and then sequentially searching for the intervention direction from the previous state point of physiological indicator parameter to the next state point of physiological indicator parameter starting from the physiological indicator parameter corresponding to the initial state point, and implementing the relevant actions specified in the corresponding intervention procedure;
step 7: after intervening with the subject for a period At according to the psychological intervention procedure corresponding to the optimal path, the data acquisition module acquires data of the subject again, and compares the data acquired at the time to the data corresponding to a corresponding standard state point on the optimal path, if the comparison of the two sets of data is within a threshold range, then continues on the intervention path of step 6, if the deviation of the two sets of data exceeds the threshold, proceeds to step 8;
step 8: calculating the distance from the deviated state point to the ideal state point and the distance from the deviated state point back to the expected state point until the ideal state point, comparing the length of the two, choosing the shortest path to continue the intervention, and repeating the method until reaching the ideal state point.

30. The psychological assessment and intervention method of claim 29, characterized in that in step 4, after the data acquisition module acquires the indicators of the subject, an arithmetic unit of the data processing module first checks the integrity of the acquired data of the subject required by each dimension, if the integrity reaches the preset standard, the arithmetic unit establishes a multi-dimensional state point of the subject, and matches the multi-dimensional state point of the subject; if the integrity does not reach the preset standard, it is required to return to the data acquisition module to continue to acquire relevant missing information until the integrity reaches the preset standard.

31. The psychological assessment and intervention method of claim 29, characterized in that, it further comprises a step 9, after the intervention procedure of step 8 is completed, the subject needs to enter an empowerment zone for the empowerment operation, the empowerment operation empowers the subject through acoustic, optical, electronic, chemical, and/or magnetic methods; wherein, the acoustic method mainly includes playing some encouraging words, playing music or special sound wave signals through microphones to effectively intervene with the subject's subconscious mind; the optical method includes embedding a specific pattern in an image or illuminate pattern to subconsciously hypnotize or induce the subject; the electromagnetic method includes transcranial electrical stimulation and transcranial magnetic stimulation, which are mainly sticking electrodes or electromagnetic units on the head in a contact or non-contact manner, sending modulated electrical or magnetic signals to the brain, so that these modulated signals affect the functioning of the cerebral cortex and affect the consciousness or mental state of the subject; the chemical method includes spreading or spraying a certain type of chemical molecules to allow the brain to generate special sensations, or induce and solidify a certain type of specific memories, taking advantage of the special position of the olfactory bulb and olfactory nerve in the brain to optimize, strengthen and even achieve effects that cannot be achieved by other intervention methods, if the released molecules are neurotransmitters (exogenous or endogenous), these chemical molecules participate in the discharge conduction function of neurons and directly change the thinking mode of the brain.
